(19) United States
(12) Patent Application Publication
(10) Pub. No.: US 2024/0016404 A1
(43) Pub. Date: Jan. 18, 2024

(54) MEASURING DEVICE FOR NON-INVASIVELY DETECTING THE INTRACRANIAL PRESSURE OF A PATIENT, AND CORRESPONDING METHOD

(71) Applicant: iNDTact GmBH, Wurzburg (DE)

(72) Inventors: Raino PETRICEVIC, Wurzburg (DE); Clemens LAUNER, Wurzburg (DE)

(21) Appl. No.: 18/256,635

(22) PCT Filed: Dec. 16, 2021
(86) PCT No.: PCT/EP2021/086314

§ 371 Date: Jun. 8, 2023

(30) Foreign Application Priority Data
Dec. 16, 2020 (DE) 10 2020 133 776.0

(51) Int. Cl. A61B 5/03; A61B 5/00

(52) U.S. Cl. CPC A61B 5/031; A61B 5/6803; A61B 5/7225; A61B 5/6831; A61B 5/742; A61B 2562/0261; A61B 2560/0214

(57) ABSTRACT

Measuring device for non-invasively detecting the intracranial pressure pulsation of a patient, comprising a holding device which can be detachably attached to the outside of the patient's skull in a force-fitting and/or form-fitting manner, at least one bimorph bending sensor arranged in or on the holding device, an analog signal amplifier for amplifying the measurement data provided by the bimorph bending sensor, an A/D converter for converting the analog measurement data into digital data, and a computing unit for preprocessing the data and calculating vital parameters such as intracranial pressure based on the digital data. In addition, an associated method for non-invasive detection of intracranial pressure pulsation is described.

The invention relates to a measuring device for non-invasively detecting the intracranial pressure of a patient.

Numerous neurointensive care conditions can be associated with a life-threatening increase in intracranial pressure (ICP). Because the volume inside the skull is constant, an increase in volume of one or more compartments can lead to an increase in ICP. These compartments include the brain tissue (e.g., due to hemorrhage, swelling, inflammation), the CSF space (e.g., due to hydrocephalus, hemorrhage), and the vascular compartment (e.g., change due to hyper- or hypoventilation). The relationship between intracranial volume and intracranial pressure is termed intracranial compliance. ICP increases exponentially with volume increase, because initially an ICP increase can be compensated by so-called reserve spaces (CSF space, vascular compartment) (Monroe-Kellie doctrine). Conditions that can lead to an increase in pressure include traumatic brain injury, epi- and subdural hematomas, space-occupying ischemic strokes, intracerebral hemorrhage, subarachnoid hemorrhage, sinus and cerebral vein thrombosis, meningitis, encephalitis, global cerebral hypoxia, and other entities such as brain tumors, intoxications, and metabolic disorders.

In order to permanently monitor the ICP in critical cases such as severe traumatic brain injury, a measurement catheter can be inserted invasively through the cranial dome. However, invasive measurement procedures are a great burden for many patients, so that monitoring is often dispensed with.

Non-invasive measurement methods have already been proposed based on the measurement of the elongation of the skull. Blood volume fluctuations due to the heartbeat cause the skull to stretch, especially across the connective tissue closed cranial sutures. The resulting pressure pulse fluctuations in the brain are approximately 3-4 mmHg. These cause a minimal pulse-synchronous expansion of the skull.

Document WO 2013/041973 A2 proposes a measuring device for non-invasive measurement of intracranial pressure, comprising a sensor designed to detect the deformation of the skull. The sensor is connected to an amplifier, an ND converter, a processor, a display, and a memory. The measuring device enables the intracranial pressure to be determined by evaluating the sensor signals, based on which the deformation of the skull can be determined.

A similar measuring device is known from WO 2019/087148 A1, in which data acquired by a sensor is sent wirelessly to a receiver after processing.

With these measuring devices, however, there is the disadvantage that a dominant influence of the pulsation of the external carotid artery cannot be excluded due to a lack of decoupling. The cranial pulsation caused by the pulsatile internal pressure, which is significantly less than the arterial pulsation, is of little importance without discrimination of the arterial pulsation. The strain gauge arrangements proposed in the cited printed materials are operated at their measurement limit. This also means that no padding of the measuring device on the skull is possible, so that prolonged use is very uncomfortable for the patient with increasing duration.

The invention is based on the task of providing a measuring device for non-invasive detection of intracranial pressure pulsation that eliminates the aforementioned disadvantages and enables simple yet reliable measurement of vital data such as static intracranial pressure.

To solve this task, a measuring device having the features of claim 1 is provided.

The measuring device according to the invention comprises a holding device which can be detachably attached to the outside of the patient's skull in a force-locking and/or form-locking manner, at least one bimorph bending sensor which is arranged in or on the holding device, an analog signal amplifier and/or an analog signal filter for amplifying and/or filtering the measurement data supplied by the bimorph bending sensor, an ND converter for converting the analog measurement data into digital data, and a computing unit for preprocessing the digital data and for calculating and/or deriving vital parameters such as intracranial pressure on the basis of the digital data.

The measuring device according to the invention is characterized by the fact that it largely eliminates the influence of pulsating arterial vessels, i.e. the intracranial pressure pulsation is actually measured. In addition to conditioning the digital data, the computing unit is also used to perform corrections and calculate characteristic curve parameters and values that can be derived from them, such as intracranial pressure (ICP). In addition, systolic or diastolic characteristic values or vital parameters can be derived from the digital data. Optionally, the measuring device according to the invention comprises a display so that amplitude curves, a measurement curve, determined parameters or derived values or warnings can be output.

The invention is based on the realization that a bimorph bend sensor can be used to provide a particularly simple yet reliable and precise measurement of intracranial pressure pulsation. The principle of the invention is based on the deflection of a bimorph bending sensor attached to a head cuff or to a contact surface on the head due to the pulse-synchronous cranial volume expansion caused by the pulsating intracranial pressure (ICP).

A piezoelectric bending sensor can measure the smallest deformations or vibrations of the skull due to the heartbeat. The pressure of the blood pumped through the heart into the brain decreases gradually during the heartbeat due to the transmission pathways through the brain tissue. The transfer function sometimes depends on the intracranial pressure and related autoregulation mechanisms, so that the dynamic course of the pressure drop can be used to infer the intracranial pressure and autoregulation status, among other things.

Preferably, the holding device is designed as a headband or head cuff. However, the holding device can alternatively also be attached by placing, gluing or clamping the bending sensor by means of a cap or bandage. The exclusive or additional use of a suitable elastic coupling medium such as a skin-friendly double-sided adhesive film is also conceivable.

The measuring device comprises the headband or cuff, which is at least partially flexurally flexible and can also be at least partially elastically stretchable. The tensioning force of the headband or cuff is adjustable. The at least one bending sensor is part of a bend-flexible portion of the headband or cuff and can bend statically or dynamically due to static or dynamic volume expansion of the skull directly or due to an associated change in tensile stress of the support attached thereto, in particular a headband. The cuff can be applied to the patient's head and secured via a tensioning device with a constant tensioning force.

Preferably, the bend sensor is a bimorph piezoelectric bend sensor. It may be a bimorph bending sensor with antiparallel polarity. In this case, use is made of the effect that the pressure pulse-induced dynamic volume deflection of a skull on which the measuring device according to the invention is arranged causes a dynamic tensile stress on the measuring device according to the invention, which is designed as a headband, and a dynamic bending at the position of the bending sensor. In this way, even extremely weak pressure-induced volume changes of the skull can be detected.

A bimorph bend sensor consists of two sensor layers symmetrically arranged around the neutral fiber. When this arrangement is bent in one direction, one of the sensory active bending sensor layers is stretched while the other is equally compressed. When bending in the other direction, the behavior is exactly the opposite. Due to an antiparallel polarity of the two sensor layers, the signals of these opposing loads, since they have the same sign, add up in terms of magnitude and increase the overall signal. Coincident effects, on the other hand, such as interfering temperature effects or pyroelectric effects, are largely cancelled out and thus compensated.

In addition to a bimorph piezoelectric bending sensor, a multimorph bending sensor can also be used, which is composed of several pairs of sensors with alternating antiparallel polarity.

It can be provided that the bending sensor is arranged like a rocker on a support which can be attached to the outside of the patient's skull. In this embodiment, the bending sensor is arranged on a support that is arranged in the region of a cranial suture of the patient. The bimorph piezoelectric bending sensor can move around a pivot point under the effect of the volume deflections of the skull like a rocker. The holding device, which is designed as a headband, generates a counter (bearing) force on the bending sensor via a defined pretensioning force, which is necessary for the bending.

In an alternative embodiment, the bend sensor is disposed on a central portion of a C-shaped holder disposed between two end portions. The C-shaped holder is placed on a patient's skull such that the two end sections are located on either side of the skull suture. Pulsatile volume deflection of the skull causes the two end sections (legs) of the C-shaped holder to move in opposite directions, resulting in bending of the middle section of the bend sensor, The attachment of the bend sensor(s) is always done in such a way that no or negligible pulsations are transmitted to the bend sensor through external arteries or veins. This can be achieved by ensuring that pulsating arteries or veins have no or only strongly damped mechanical contact with the bend sensor or its attachment, i.e. with the headband. The C-shaped holder of the measuring device according to the invention allows strongly pulsating external vessels such as large arteries to be effectively and easily bridged for this purpose. This can additionally be done at other locations by using recesses in the support. By using foam padding, the influence of smaller and thus weaker pulsating external vessels can also be effectively damped below the influence limit. A direct or insufficiently damped contact to a pulsating artery would be immediately visible in the time signal by means of the typical “arterial curve shape” and the significantly higher amplitude.

Another alternative embodiment provides for the C-shaped holder to be fastened around the head with a cuff or strap like a headband, so that the pulsating volume deflection is transmitted to the tensile stress of the cuff or strap and this in turn causes a corresponding bending via the legs of the C-shaped holder, which is detected by the bending sensor. Via such a cuff, the smallest volume deflections of the skull can be transmitted in the form of a tensile stress to the C-legs of the sensor holder. This also results in a bending of the C-arm, which is detected by the bending sensor.

A defined pretensioning force is generated by such a headband. The change in volume of the skull caused by the pulsations of the intracranial pressure cause a bending of the piezoelectric bending sensor, which can be detected by means of the measuring device according to the invention. Using the measured values obtained in this way, the intracranial pressure pulsation and thus its pressure pulse shape can be monitored and vital state variables such as intracranial pressure can be calculated from the pressure pulse shape characteristics and its characteristic values. The C-shaped holder can also be arranged inverted on the patient's skull, i.e., so that the two end sections extend away from the skull. In this case, the headband also creates a pretensioning force. The bimorph bending sensor can be placed on either side or symmetrically on both sides of the middle section. Alternatively, in this example as in all other examples, a bimorph bend sensor may be placed in the neutral fiber of the center section. It is also possible for multiple piezoelectric bend sensors to be arranged inside the middle section symmetrically with respect to the neutral fiber. A soft, elastic support pad can also be placed on the outside of the skull, onto which the C-shaped holder is attached. Alternatively, the pad may be attached to the headband in such a way that it can be quickly replaced.

Preferably, the holding device designed as a headband can have a device for generating and adjusting a pretensioning force acting on the patient's skull. Preferably, the device for generating the pretensioning force may comprise a force sensor or a strain sensor. The pretensioning force may be adjusted by a user via a hand wheel or the like, or alternatively by means of a motor. For this purpose, the device may include a linear-elastic strain element such as a tension spring. In a further embodiment, this linear-elastic expansion element can be fixed, i.e. blocked, with respect to a further deflection after a constant tensile stress has been set.

In this context, it is preferred that the device for generating the pretensioning force has an indicator for the pretensioning force or for a tensions assigned to it. In this way, the user can set and control a specific pretensioning force that is transmitted to the piezoelectric bending sensor via the headband.

To further simplify the use of the measuring device according to the invention, the device for generating the pretensioning force can be designed to automatically set a predefined pretensioning force. An electromechanical or a pneumatic mechanism may be provided for this purpose. Manual or automatic control of the pretensioning force can be achieved by pneumatic tensioning force adjustment using an integrated air cushion in combination with an air pressure sensor.

Optionally, the headband may have padding over at least part of its length. The padding may also consist of several separate padded support points. The padding is located on the inside of the support device configured as a headband. The padding may comprise an elastic foam or a viscoelastic memory foam. The headband or cuff may be in full contact or only in defined contact areas or contact points to minimize interference from pulsatile soft tissues such as peripheral blood vessels and muscle activity, or to avoid contact with injuries.

It can also be provided that the measuring device according to the invention has one or more structure-borne sound sensors and/or one or more acceleration sensors, one or more position sensors and/or one or more pulse sensors and/or one or more blood pressure sensors and/or a temperature sensor, and that the computing unit is designed to detect external disturbing influences by at least one of the sensors mentioned. These external disturbing influences can be eliminated by calculation after detection so that they do not adversely affect the measurement of the intracranial pressure.

Preferably, the bending sensor can be removed from the holder designed as a headband and replaced. Retaining devices such as recesses and/or retaining clips are provided at the preferred sensor position of the headband or the cuff for fastening the sensor so that a form fit and/or a force fit is made possible. However, the bend sensor may also be bonded and/or screwed to the headband or cuff. The headband can be reused for another patient after sterilization. It is also possible that the headband has different positions for attaching the bend sensor. It is also possible that several bend sensors are attached to the headband.

One embodiment of the measuring device according to the invention provides that the bimorph bending sensor and the analog signal amplifier are integrated in a single component. Optionally, the following components may also be integrated in the single component: ND converter, a transmitting device, a transmitting-receiving device for wireless data transmission, a battery, a rechargeable battery. This reduces the number of components and the measuring device requires only a small installation space.

It may also be provided that the measuring device has a data logger connected to the A/D converter or the computing unit. The data logger stores either the measured values of the bending sensor and/or the data derived therefrom, such as the intracranial pressure. The data stored in the data logger can thus also be evaluated at a later time. The measuring device according to the invention can thus also be designed as a mobile device.

The holding device arranged as a headband may include an energy storage device, preferably a battery or a rechargeable battery, thereby enabling use as a mobile device.

Further possible applications arise if the piezoelectric bending sensor and/or the analog signal amplifier and/or the A/D converter is/are connected to a transmitting device or a transmitting/receiving device for wireless data transmission. In this case, data acquired by the sensor can be transmitted to a receiver, optionally after amplification or after conversion to digital data. In the case of wireless data transmission, the holding device designed as a headband does not require any cable connections, which simplifies and facilitates handling.

A variant of the measuring device according to the invention provides that several piezoelectric bending sensors are arranged on the headband. This makes it possible to measure the intracranial pressure pulsation and thus the intracranial pressure at several points.

In addition, the invention relates to a method for non-invasively detecting the intracranial pressure pulsation of a patient with a measuring device of the type described having the features of claim 17. The method according to the invention comprises the following steps: force-locking and/or form-locking attachment of the holding device, which has the at least one bimorph bending sensor and is in the form of a headband, to the outside of the patient's skull, dynamic detection of deformations and/or oscillations of the skull caused by the person's heartbeat by means of the at least one bending sensor, and calculation of characteristic curve parameters on the basis of the deformations and/or oscillations of the skull detected by means of the bending sensor and on the basis of a measured pulse curve, and derivation of vital state variables such as the intracranial pressure (ICP).

The method may also include the following steps: Digitization, signal preprocessing, determination of characteristic parameters. In the method according to the invention, it is preferred that the dynamically recorded deformations and/or oscillations of the skull are used to measure the “pressure response function” due to the cardiac pulsation excitation and to calculate therefrom an intracranial pressure and other parameters that are related to various vital state variables. For example, the procedure can be performed for the following diseases or conditions: head trauma, vasospasm, infarction, occlusions, reperfusion, revascularization, tension headache, migraine, embolus detection with carotid stenosis, dementia, hydrocephalus, brain tumor, sickle cell anemia, vascular malformations, meningitis, encephalitis, coma, heart failure, Aortic stenosis, aortic regurgitation, aortic valvuloplasty, carotid revascularization, aortic dissection, cardiopulmonary bypass, anesthesia, hyperventilation, catecholamines, volume management, hemofiltration, hemodialysis, pulmonary arterial hypertension, renal insufficiency, hemodialysis, peritoneal dialysis.

A variant of the method according to the invention provides that the detection of the pulsation of the cranial deflection due to the intracranial pressure pulsation and/or its effects is performed with two or more bending sensors arranged frontally at the base of the skull.

Alternatively or additionally, the detection of intracranial pressure can be performed with two or more bending sensors located occipitally at the base of the skull.

Preferably, the method according to the invention is carried out permanently, with vital parameters and intracranial pressure being recorded or derived at fixed intervals. In this way, long-term monitoring of a patient is also possible.

In the method according to the invention, the at least one bending sensor can be attached to the skull by placing, gluing or clamping. Preferably, the holding device designed as a headband is used for this purpose.

It is also possible that the at least one bending sensor is connected to the skull as an inlay of an exoskeleton or a helmet. This ensures a uniform contact pressure of the sensor or sensors.

The invention also includes a computer program suitable for the following functions:
Detection of onset (beginning and end) of individual pulse courses (pulse curve),
Detection of interfering signals (by coughing, speaking, movement, etc.)
Discrimination of non-evaluable trajectories (e.g., due to interference),
Evaluation of curve trajectories using supervised (e.g. trained neural networks) and/or unsupervised (e.g. cluster analysis) machine learning programs (artificial intelligence),
Detection of other vital signs (respiration, blood pressure, mood, . . . ),
Correction or filtering of signal drift overlays (e.g. due to respiration),
Determination of the number, position and amplitude of curve characteristics such as peaks, notches and inflection points from individual pulse curves,
Determination of characteristic parameters describing the course of the curve,
Determination of statistical data (mean values, distribution, dispersion, trends) of the curve characteristics,
Determination of the curve area under the drift-corrected pulse curve or specified sections thereof, in particular the systolic and diastolic areas,
Distinguish between systolic and diastolic curve sections when determining parameters,
Formation of any relations between two or more of the parameters determined from the curve or from individual curve sections.

### EXAMPLES

P₂/P₁, P₂/P₁, Aₜₒₜₐₗ, P₁²-P₃²|, P₁/P₃, |P₁²-P₃²|, Aₜₒₜₐₗ, A_{sys}/A_{dia},
Establishing various relations between at least one of the parameters and/or their relations to other medical metrics (blood pressure, pulse, blood values, body temperature, . . . ) and patient parameters (age, gender, weight, height, ancestry, skull geometry, athleticism, . . . ),
Mean values and statistical evaluation (distribution function parameters) of these quantities from the evaluation of several pulse curves,
Trend curves of individual parameters or their relations to each other,
Derivation of diagnostic variables such as intracranial pressure (ICP), cerebral blood flow (CBF), cerebral perfusion pressure (CPP), cerebrovascular resistance (CVR), arterial and mean arterial pressure ((M)AP), pulsatility index (PI), resistance index (RI), systolic and diastolic pressure S/DP, systolic and diastolic pressure-time index (SPTI, DPTI) ,
Derivation of autoregulatory disorders or abnormalities,
Derivation of typical disease characteristics from above parameters and relations,
Derivation of infections,
Derive correlations to various health conditions or diseases, physical and mental stress states, relaxation states, external influences, with other vital parameters, exercise performance, medication intake, workload, mental illness.

Due to the extraordinarily high signal quality, especially the diastolic pressure curve range can be evaluated for itself and derivations to the entire field of application can be performed.

Clear curve characteristics can be obtained from the diastolic pressure curve area using the measuring device of the invention, in contrast to other methods such as transcranial Doppler, from which, for example, a diastolic flow velocity profile is obtained. These contain important information about effects of changes, diseases, etc., that can be attributed to microcirculatory disturbances, increased tissue resistance, intracranial pressure elevation, chronic inflammation, arteriosclerosis, diabetes, and inadequate O₂ or CO₂ exchange, hypotension, or hypovolemia.

Thus, pathological changes in these areas can be detected early without an invasive procedure and progressive changes can be monitored easily. In addition, treatment effects in such areas can be assessed much more easily and therapies can be directed more specifically.

The invention is explained below by means of embodiment examples with reference to the drawings. The drawings are schematic representations and show:

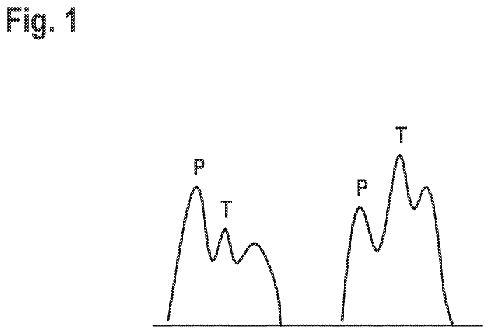

FIG. 1A normal course and a pathological course of intracranial pressure over time;

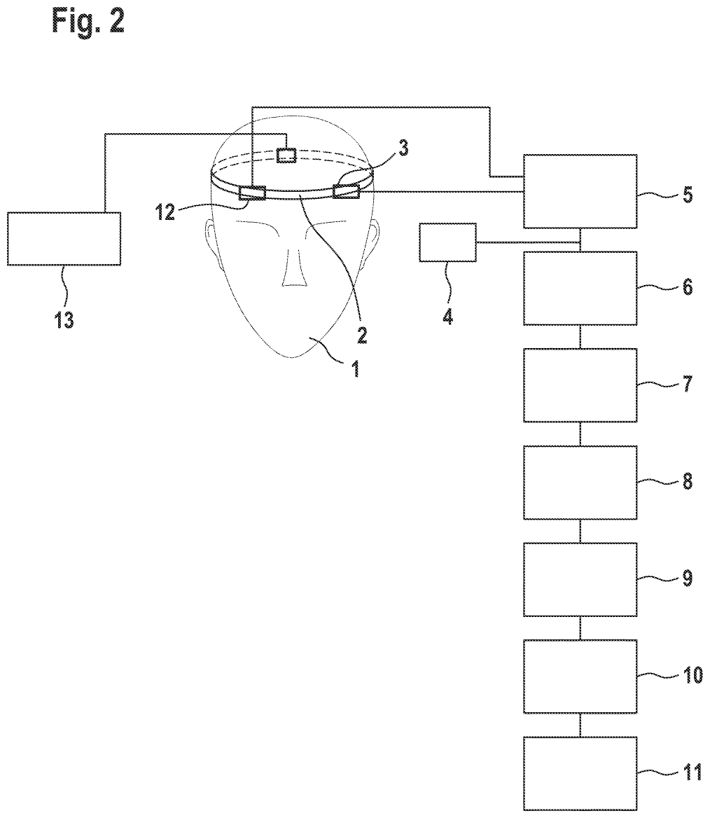

FIG. 2 the essential components of a measuring device according to the invention;

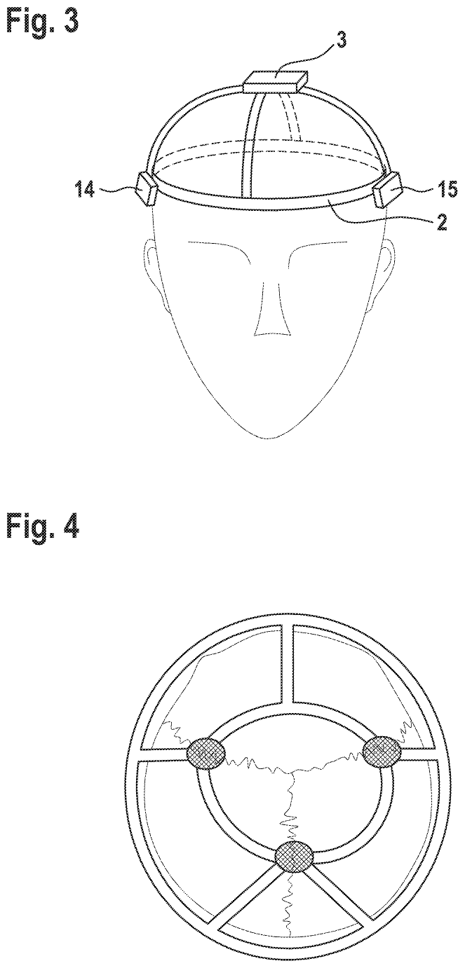

FIG. 3 another embodiment of a holding device designed as a sleeve;

FIG. 4 an example of a holding device with several bending sensors;

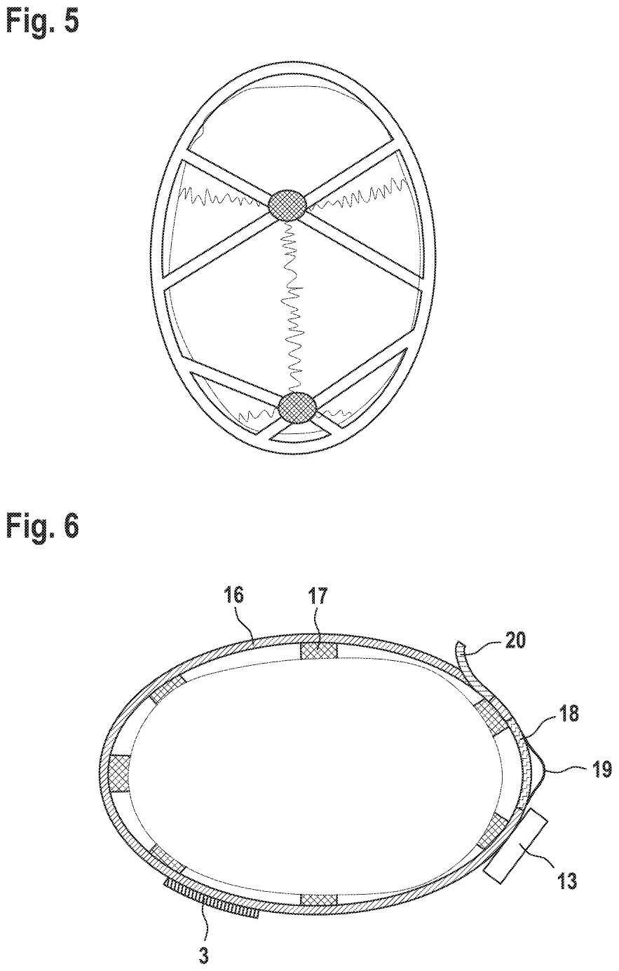

FIG. 5 another embodiment of a holding device with multiple bending sensors;

FIG. 6 a top view of a holding device designed as a sleeve;

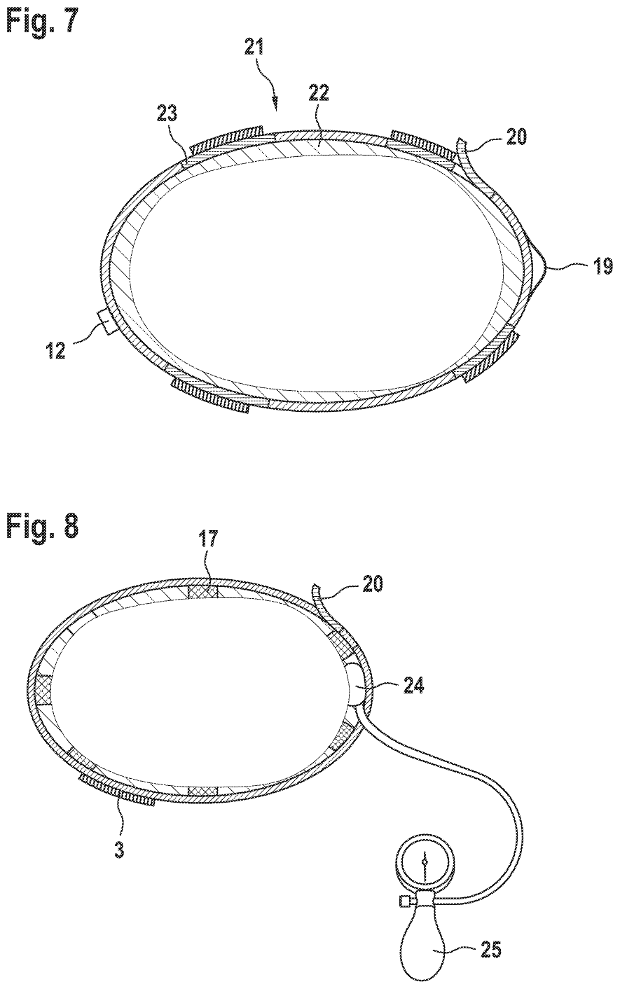

FIG. 7 a further example of a retaining device in the form of a sleeve in a plan view;

FIG. 8 a similar embodiment of a sleeve as FIG. 6;

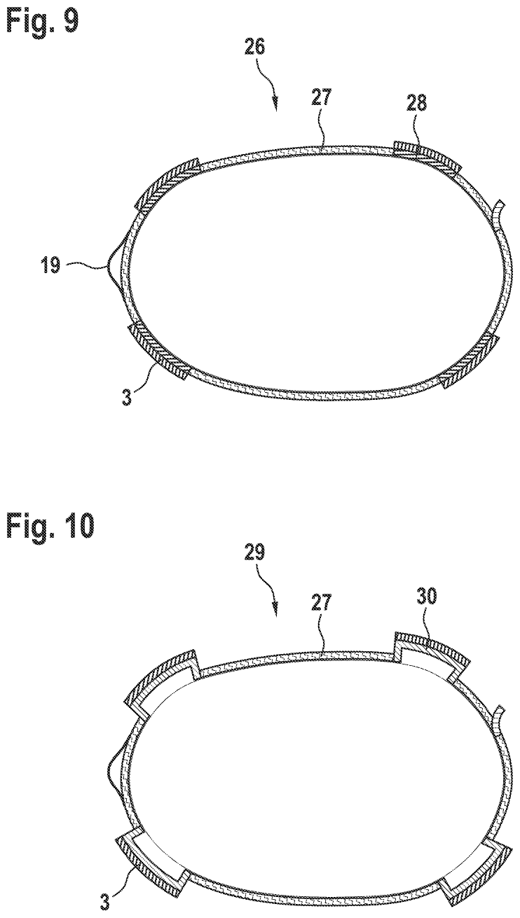

FIG. 9 an embodiment of a cuff with an expandable band;

FIG. 10 an embodiment of a cuff with a stretchable elastic band;

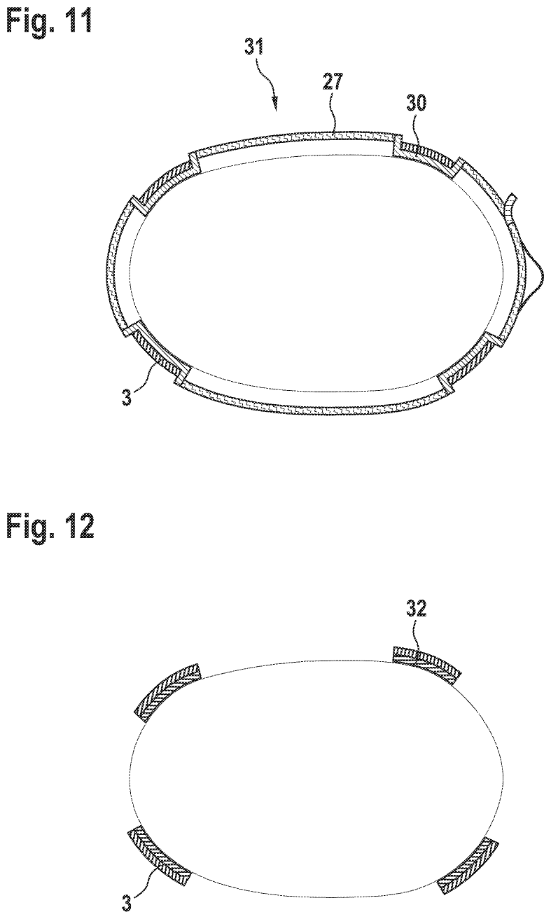

FIG. 11 another embodiment of a cuff;

FIG. 12 a measuring device without cuff or tape;

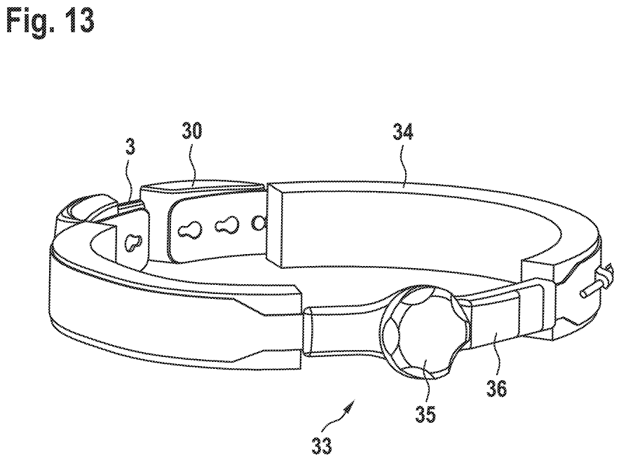

FIG. 13 a measuring device with a cuff;

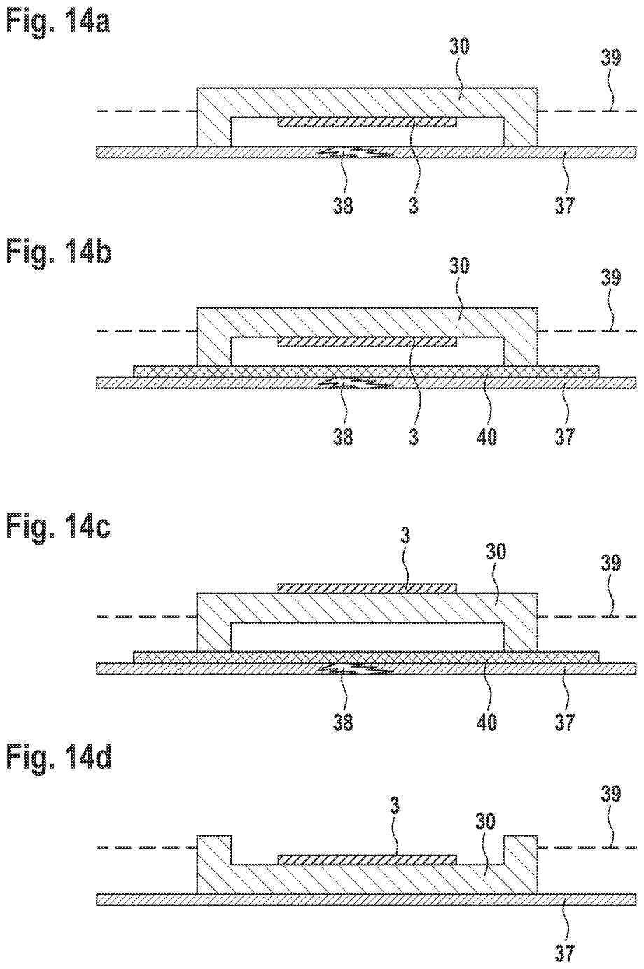

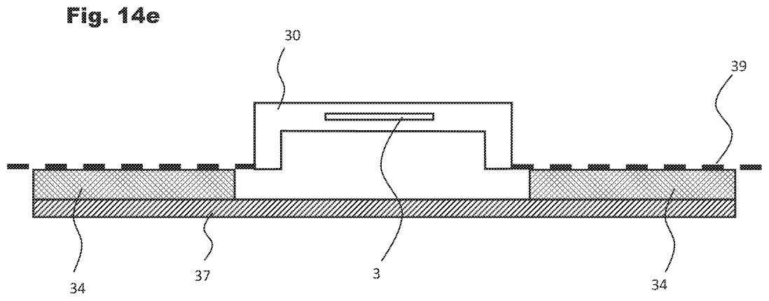

FIG. 14a-14e different versions of a C-shaped holder;

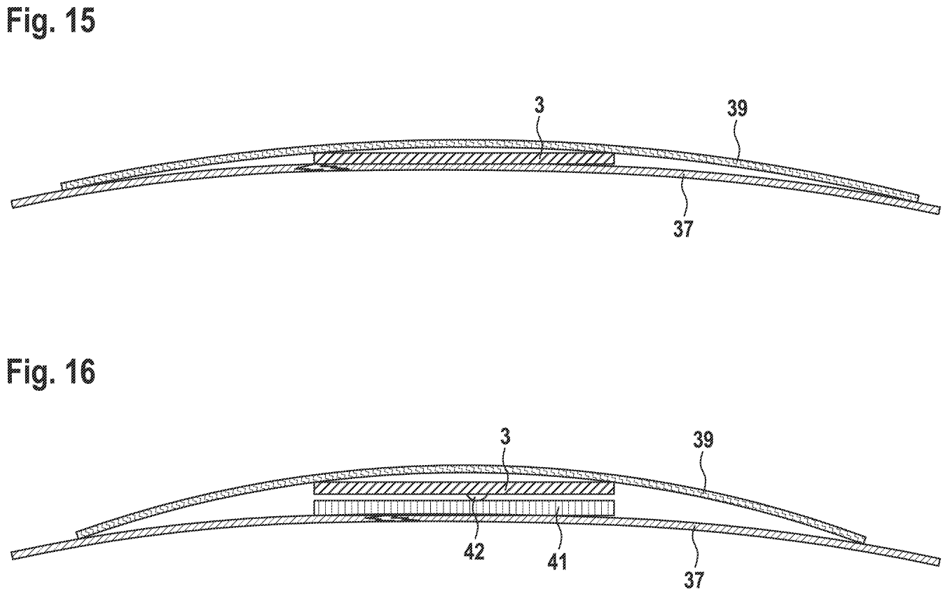

FIG. 15 a cutaway view of a bending sensor placed on a skull;

FIG. 16 a bending sensor placed on a skull in a sectional view,

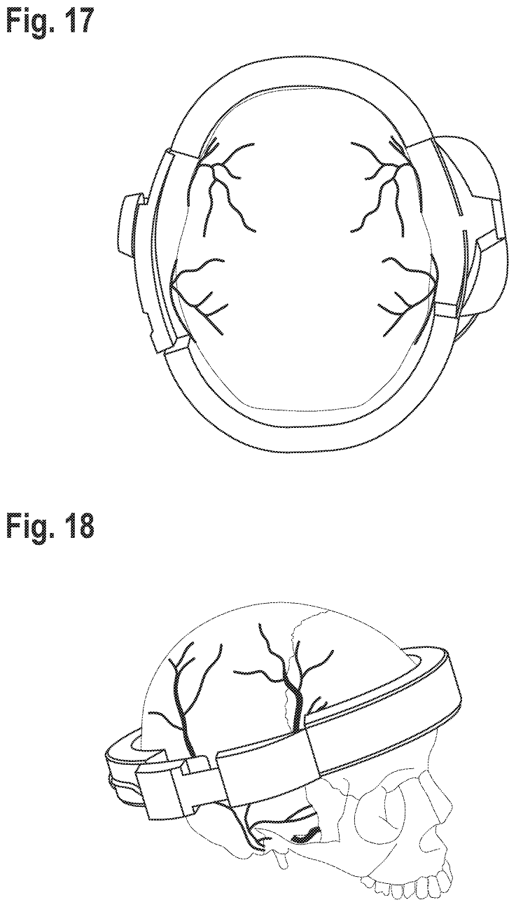

FIG. 17 a top view of a holding device placed on a skull;

FIG. 18 a view of the right side of the holding device shown in FIG. 17, and

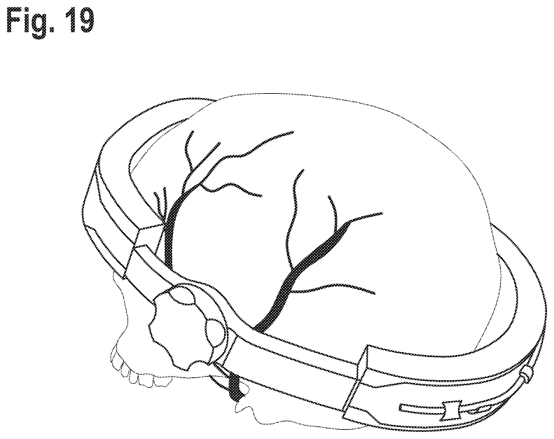

FIG. 19 a view of the left side of the holding device shown in FIG. 17.

The left part of FIG. 1 shows qualitatively a normal course of the intracranial pressure, the right part of FIG. 1 shows a pathological course of the intracranial pressure. Time is plotted on the horizontal axis, and an electrical voltage detected by the sensor is plotted on the vertical axis. Based on the waveform of the voltage-time signal, the intracranial pressure can be determined. Characteristic values for this are, for example, the rise quotient (U1−U0)/t0, the number of peaks per cardiac cycle, which can typically be 3 to 6. The evaluation can also be performed on the basis of distances between prominent points of at least one pulse signal and QRS components recorded in parallel by means of an electrocardiogram or an external arterial pulse signal tapped at the head, neck, arm or finger. For evaluation, correlation or correction with the patient's pulse rate or respiratory rate can also be performed.

With continuous recording, intracranial pressure (ICP) shows a multipeak pulse-synchronous periodic course: The first peak P is caused by the main arterial pressure wave. A second peak T is caused by filling of the cerebral arteries with blood and depends on intracranial compliance. A third peak or even several further peaks are related to diastolic-induced pulsations, e.g., closure of the aortic valves.

With increasing ICP, T increases relative to P as well as the total pulse pressure amplitude, so that the curve shape becomes increasingly pyramidal. From the dynamic course, it is thus possible to draw a conclusion about an increased static intracranial pressure.

By detecting the deflection of a bimorph piezoelectric bending sensor attached to a head cuff, headband, or support surface on the head due to pulse-synchronous cranial volume expansion caused by intracranial pressure pulsating at approximately 3-4 mmHg, mean static intracranial pressure (ICP) can be indirectly determined from the pulse shape. Absolute measured blood pressure values can be added to increase the accuracy of this procedure.

Essential components of the measuring device or steps of the measuring procedure are explained with reference to FIG. 2. A holding device 2 is attached to the head of a person 1, which is designed as a headband or cuff. A piezoelectric bending sensor 3 is located on the headband, which is detachably arranged on the outside of the head of the person 1. Associated with the bending sensor 3 is an energy storage device in the form of an energy storage 4. In addition, the measuring device comprises an analog signal amplifier 5 with an analog filter. This is followed by an ND converter 6 which converts the analog signals into digital data. In a filter 7, filtering of the digital data, smoothing and data reduction take place. The measuring device further comprises an interface 8 for transmitting signals or data. The signals or data can be transmitted, for example, to an external device, a computing unit or an evaluation unit. The data are used to determine characteristic values which are stored in a memory 9 for characteristic values. A valuation unit 10 evaluates the data or characteristic values. A display 11 is used to output measured data and other information. This includes recorded measured values, signals, characteristic values, an evaluation or a warning. A structure-borne sound sensor 12 is also attached to the holding device 2 for detecting interference signals. This way, interfering signals caused by external signal sources can be eliminated. A component of the measuring device is also a device 13 for generating a pretensioning force. By means of the device 13, a defined pretensioning force applied to the bending sensor 3 can be generated.

FIG. 3 shows an example of an embodiment in which the holding device 2, designed as a sleeve, has two further bending sensors 14, 15 in addition to the components shown in FIG. 2. The bending sensor 3 arranged on the head contains the further components mentioned in FIG. 2, such as an energy storage device, an analog signal amplifier, an ND converter, etc.

FIGS. 4 and 5 show further examples of holding devices, each having multiple bending sensors that are temporarily fixed to a patient's skull by applying a pretensioning force acting on the bending sensor.

FIG. 6 shows a schematic top view of a holding device designed as a cuff 16, which is attached to a skull. The cuff 16 has several spaced pads 17 on its inner side, each of which forms a contact surface on the skull. There may be a space between adjacent pads 17, or alternatively the space may be filled by foam. A bending sensor is arranged on the outside of the cuff 16. The cuff 16 also comprises the device 13 for generating a pre-tensioning force and an elongation element 18 formed as a rubber band. The elongation is limited by an elongation limiter 19. The cuff 16 also includes a hook-and-loop fastener 20 for securing a free end of the cuff 16.

In an alternative embodiment, the cuff can be provided on its inside with a viscoelastic memory foam. This has the property that it becomes hard under rapid loading, especially in the event of a rapid impact.

FIG. 7 shows a further embodiment of a retaining device designed as a cuff 21. The cuff 21 is made of an elastic, i.e. stretchable, material. In accordance with the preceding embodiment, the cuff 21 has the hook-and-loop fastener 20 and the elongation limiter 19. On the inner side of the cuff 21, there is a viscoelastic memory foam 22 as a cushion. A total of four bending sensors 3 distributed around the circumference are arranged on the outside of the cuff 21. Each bending sensor 3 is mounted on a flexible pad 23. In addition, a structure-borne sound sensor 12 is arranged on the outside of the cuff 21.

FIG. 8 shows an embodiment similar to the cuff 16 shown in FIG. 6. In addition to the pads 17, which form contact surfaces, and the bending sensor 3, the cuff has an air cushion 24 that can be inflated by a manually operated pump 25.

The described embodiments each show closed cuffs that extend over the entire circumference of a patient's skull. However, a cuff may also extend and be clamped over only a portion of the circumference of the skull. For this purpose, the cuff can be made of a flexible material, a bendable material or a spring-elastic material.

FIG. 9 shows an embodiment of a cuff 26 having a stretchable elastic band 27 extending around the entire circumference of a skull. On the outside of the elastic band 27 are a plurality of bending sensors 3, which are attached to the skull of a patient via a bending flexible element 28 with a pad. The elastic band 27 also has an elongation limiter 19.

In a modified embodiment, a non-stretchable tension strap may be used instead of an expandable rubber band. In this case, a short stretchable element is required to attach the cuff to a skull with some pretension.

FIG. 10 shows an embodiment of a cuff 29 having a stretchable elastic band 27 and a plurality of bending sensors 3, each arranged on the outside of a C-shaped holder 30. A C-shaped holder 30 includes a central portion and two end portions extending perpendicularly therefrom. The end portions of the C-shaped holder 30 face the skull. The C-shaped holders 30 are flexurally flexible (bendable) and are arranged circumferentially on a skull of a patient so as to cover a cranial suture. A pulsating elongation of the skull can be detected by means of the bending sensors 3.

FIG. 11 shows an embodiment of a cuff 31 similar to the embodiment shown in FIG. 10. A total of four C-shaped holders 30 are arranged on the cuff 31, the end portions of which face away from the cranial path. A bending sensor 3 is located on the outside of each C-shaped holder 30.

FIG. 12 shows an example of a measuring device in which the holding device is designed as a bending flexible element 32. A bending sensor 3 is arranged on the outside of the bending flexible element 32. A total of four such bending sensors 3 are present over the circumference of the skull. The bending flexible elements 32 are bonded to the skull, and a sleeve or tape is not required in this embodiment.

FIG. 13 shows a measuring device with a cuff 33, a C-shaped holder 30 and a piezoelectric bending sensor 3. On the inside of the cuff 33 there are pads 34 made of foam. On the side opposite to the piezoelectric bending sensor, there is a device 13 for generating a pretensioning force with an adjusting screw 35. The device 13 includes an indicator 36 for indicating the pretensioning force.

FIGS. 14a to 14e show various embodiments of a C-shaped holder.

In FIG. 14a, it can be seen that the C-shaped holder 30 is arranged with its end sections on the outer surface 37 of a patient's skull. The bending sensor 3 is located on the inner side of the C-shaped holder 30, which is arranged to cross a skull suture 38. A tape 39 is used to secure the C-shaped holder 30 in place. The tape 39 may be rigid, flexible, bendable, or stretchable.

FIG. 14b is a similar view to FIG. 14a, with a soft elastic pad 40 disposed between the outer surface 37 of the skull and the C-shaped holder 30.

FIG. 14c is a similar view to FIG. 14b, with the bending sensor 3 located on the outside of the C-shaped holder 30.

FIG. 14d shows an embodiment in which the C-shaped holder 30 rests with its central portion on the outer surface 37 of a skull. The end sections of the C-shaped holder 30 thus protrude from the outer surface 37 of the skull. The bending sensor 3 is located on the outer side of the C-shaped holder 30, which is held by the tape 39. Optionally, a soft elastic pad can be arranged between the outer surface 37 of the skull and the C-shaped holder 30.

FIG. 14e shows an embodiment in which the bending sensor 3 is integrated into the holder 30. It is located inside the middle section of the holder 30, which can be attached anywhere on the outer surface 37 of a patient's head, but in such a way that arteries are bridged in the process. A pad 34 is located between the skull and the angled end sections, and a pretensioning force can be introduced by a strap or tape 39 attached to either side of the end sections of the C-shaped retainer.

FIG. 15 is a cutaway view and schematically shows a bimorph piezoelectric bending sensor 3 placed on the outer surface 37 of a skull. The tape 39 is used to generate a pretensioning force. One end of the bending sensor 3 is placed on a cranial suture, which may be, for example, the sutura coronalis, the sutura sagittalis or the sutura lambdoidea.

FIG. 16 is a cutaway view and shows a bimorph piezoelectric bending sensor 3 with a convex, outwardly curved projection 42 at the center of its underside, which rests on a rigid support 41. The support 41 covers a skull seam. A preload acting on the bending sensor 3 is generated by the tape 39. The projection 42 supports the bending sensor 3 like a rocker, and changes in the volume of the skull can be transmitted to the sensor via the band, causing it to bend and thus be detected.

FIG. 17 is a top view and shows a holding device arranged on a skull. FIG. 18 shows the right side of the holding device shown in FIG. 17 with the C-shaped holder and FIG. 19 shows the left side of the holding device shown in FIG. 17 with the locking device with which a pretensioning force can be generated.

In FIGS. 17-19, it can be seen that the C-shaped holder of the holding device is arranged with its end sections on the outside of a patient's skull. The bimorph bending sensor is located symmetrically with respect to the neutral fiber of the middle section of the C-shaped holder inside this holder, which is attached to the skull so as to bridge the external carotid artery. A ribbon is used to transmit the volume deflection of the skull caused by intracranial pressure pulsation into a bend of the C-shaped holder. The ribbon of this retainer is rigid in the direction of traction, flexible in the direction of bending, and padded toward the skull. On the opposite left side is the locking device, which is used to adjust the pretension, as well as an indicator for the pretension. The locking device is also C-shaped and bridges the external carotid artery to prevent interference caused by it.

By selectively mechanically coupling the ribbon or padding to one or more arteries (e.g., carotid artery), the arrangement can also be used to measure blood pressure pulsation and thus blood pressure measurement at the head. In this configuration, the signal from external blood pressure pulsation is significantly higher than the signal from cranial deflection caused by intracranial pulsation. Such “transient” coupling can be achieved by rotating the head cuff, i.e., the ribbon, by 90° so that the carotid artery is below the bearing surfaces of the ribbon. However, the same could be done without rotating the ribbon by inserting a foam under the locking mechanism and/or the C-shaped support. Advantageously, the head cuff formed as a ribbon contains a coupling element at the position of the C-shaped holder and/or the locking mechanism, which can be reversibly coupled to the carotid artery. This can be done, for example, via a thread or a deployable coupling mechanism analogous to a snap-action switch. The coupling force can be adjusted with the existing locking device, preferably to a value at which the system has been calibrated.

The described features of the measuring device and the associated method can be combined in any way.

### LIST OF REFERENCE SIGNS

1 head
2 holding device
3 bending sensor
4 energy storage
5 analog signal amplifier
6 ND converter
7 filter
8 interface
9 memory
10 valuation unit
11 display
12 structure-borne sound sensor
13 device for generating a pretensioning force
14 bending sensor
15 bending sensor
16 cuff
17 pad
18 elongation element
19 elongation limiter
20 hook-and-loop fastener
21 cuff
22 memory foam
23 flexible pad
24 air cushion
25 pump
26 cuff
27 elastic band
28 bending flexible element
29 cuff
30 C-shaped holder
31 cuff
32 bending flexible element
33 cuff
34 pad
35 adjusting screw
36 display
37 outer surface
38 skull suture
39 tape
40 pad
41 support
42 projection

1. Measuring device for non-invasively detecting the intracranial pressure pulsation of a patient, comprising:
a holding device which can be detachably attached to the outside of the patient's skull in a force-locking and/or form-locking manner,
at least one bimorph bending sensor arranged in or on the holding device,
an analog signal amplifier for amplifying the measurement data supplied by the bimorph bending sensor,
an ND converter for converting the analog measurement data into digital data, and
a computing unit for preprocessing the data and calculating parameters from the intracranial pressure pulsation curve which correlate with vital state variables on the basis of the digital data.

2. Measuring device according to claim 1, wherein the holding device is formed as a headband or head cuff and/or has a display to display a measurement curve, a calculated parameter and an associated time course.

3. Measuring device according to claim 1 or 2, wherein the bimorph bending sensor is a piezoelectric bimorph bending sensor.

4. Measuring device according to any of the preceding claims, wherein the bending sensor is arranged like a rocker on a support which can be attached to the outside of the patient's skull.

5. Measuring device according to any one of claims 1 to 3, wherein the bending sensor is arranged at or in a middle section of a C-shaped holder arranged between two end sections.

6. Measuring device according to any one of claims 2 to 5, wherein the holding device designed as a headband has a device for generating and adjusting a pretensioning force acting on the patient's skull, the device preferably having a force sensor or a strain sensor.

7. Measuring device according to claim 6, wherein the device for generating the pretensioning force comprises an indicator for the pretensioning force or a voltage associated therewith.

8. Measuring device according to claim 6 or 7, wherein the device for generating the pretensioning force is designed for automatically setting a predetermined pretensioning force and preferably comprises an electromechanical or a pneumatic mechanism.

9. Measuring device according to any of the preceding claims, wherein the headband has pads over at least part of its length.

10. Measuring device according to any of the preceding claims, wherein the device comprises one or more of the following sensors: structure-borne sound sensor, acceleration sensor, position sensor, external pulse sensor, external blood pressure sensor, temperature sensor, and wherein the computing unit is designed to detect external disturbing influences or conditions detected by at least one of said sensors and, if necessary, to correct disturbing influences.

11. Measuring device according to any of the preceding claims, wherein the bending sensor is removable and replaceable from the holder formed as a headband.

12. Measuring device according to any of the preceding claims, wherein the measuring device comprises a data logger connected to the ND converter or the computing unit.

13. Measuring device according to any of the preceding claims, wherein the holding device arranged as a headband comprises an energy storage device, preferably a battery or a rechargeable battery.

14. Measuring device according to any of the preceding claims, wherein the piezoelectric bending sensor and/or the analog signal amplifier and/or the ND converter are connected to a transmitting device or a transmitting-receiving device for wireless data transmission.

15. Measuring device according to any of the preceding claims, wherein the bimorph bending sensor and the analog signal amplifier and the ND converter and/or the transmitting device, if any, and/or the transmitting-receiving device for wireless data transmission, if any, and/or the battery or rechargeable battery, if any, are integrated in a single component.

16. Measuring device according to any of the preceding claims, wherein a plurality of piezoelectric bending sensors are arranged on the headband.

17. A method for non-invasively detecting intracranial pressure pulsation of a patient with a measuring device according to any one of claims 1 to 16, comprising the following steps:
force-locking and/or form-locking attachment of the holding device comprising the at least one bimorph bending sensor to the outside of the patient's skull,
dynamic detection of deformations and/or vibrations of the skull caused by the heartbeat of the person by means of the at least one bending sensor, and
calculating characteristic measurement curve parameters on the basis of the deformations and/or vibrations of the skull recorded by means of the bending sensor and on the basis of a measured blood pressure and/or pulse curve,
derivation of vital state or diagnostic variables from characteristic waveform parameters such as intracranial pressure (ICP), cerebral blood flow (CBF), cerebral perfusion pressure (CPP), cerebrovascular resistance (CVR), arterial and mean arterial pressure ((M)AP), pulsatility index (PI), resistance index (RI), systolic and diastolic pressure S/DP, systolic and diastolic pressure-time index (SPTI, DPTI).

18. Method according to claim 17, wherein the time course of characteristic curve parameters and/or vital state variables derived therefrom is displayed.

19. Method according to claim 17 or 18, wherein a holding device designed as a headband or head cuff is used.

20. Method according to any one of claims 17 to 19, wherein the evaluated parameters are related to various neurological, cardiological, intensive care, pulmonological as well as nephrological vital states on the basis of the dynamically recorded deformations and/or vibrations.

21. Method according to any one of claims 17 to 20, wherein pathological and/or progressive changes due to systolic impairments such as reduced blood supply and reduced oxygen supply in the brain and due to diastolic impairments such as reduced cerebral perfusion and reduced oxygen supply are monitored on the basis of the dynamically recorded deformations and/or oscillations and the curve parameters derived therefrom.

22. The method of any one of claims 17 to 21, wherein the sensing is performed with two or more bending sensors located frontally at the base of the skull.

23. The method of any one of claims 17 to 22, wherein the sensing is performed with two or more bending sensors arranged occipitally at the base of the skull.

24. Method according to any one of claims 17 to 23, wherein it is performed permanently, wherein the intracranial pressure is recorded at fixed time intervals.

25. The method of any one of claims 17 to 24, wherein the at least one bend sensor is attached to the skull by layup, bonding, or clamping.

26. Method according to any one of claims 17 to 25, wherein the at least one bending sensor is connected to the skull as an inlay of an exoskeleton or a helmet.
